(19) United States
(12) Patent Application Publication
(10) Pub. No.: US 2011/0318331 A1
(43) Pub. Date: Dec. 29, 2011

(54) Nanoparticles and Use Thereof

(75) Inventors: Chih-Kuang Wang, Kaohsiung City (TW); Gwo-Jaw Wang, Taipei City (TW); Mei-Ling Ho, Kaohsiung City (TW); Cherng-Chyi Tzeng, Kaohsiung City (TW); Je-Ken Chang, Kaohsiung City (TW); Yin-Chih Fu, Kaohsiung City (TW)

(73) Assignee: KAOHSIUNG MEDICAL UNIVERSITY, Kaohsiung City (TW)

(21) Appl. No.: 12/825,648

(22) Filed: Jun. 29, 2010

(51) Int. Cl. A61K 47/30; C07K 2/00; C08G 63/00; C08G 63/06

(52) U.S. Cl. 424/130.1; 525/450; 530/300; 525/410; 525/418; 514/44 R; 514/1.1; 424/174.1

(57) ABSTRACT

A novel nanoparticle and use thereof were provided in the present invention. In particular, the nanoparticle is used for delivering therapeutic component, such as oligonucleotide and hydrophobic drug.

### FIELD OF THE INVENTION

The present invention relates to therapeutic compound delivery carriers. In particular, the carriers are cationic nanoparticles.

### BACKGROUND OF THE INVENTION

Drug delivery is the method or process of administering a pharmaceutical compound to achieve a therapeutic effect in humans or animals. Over the years, numerous methods have been proposed for administering biologically-effective materials. Medicinal agents are quite often insoluble in aqueous fluid or rapidly degraded in vivo.

For example, alkaloids are often difficult to be solubilized and proteins are often prematurely degraded upon administration into the body. One of the attempts to solve the obstacles is to include such medicinal agents as part of a soluble transport system. Such transport systems can include permanent conjugate-based systems or prodrugs.

In particular, polymeric transport systems can improve the solubility and stability of medicinal agents. Multifunctional therapeutics such as proteins can be employed in permanent conjugate-based transport systems including polymers. Proteins employed in such systems maintain biological activities to achieve therapeutic effects.

Gene therapy has gained significant attention over the past two decades as a potential method for treating genetic disorders. One of research directions was focused on designing effective carrier vectors that compact and protect oligonucleotides for gene therapy. In these gene vectors, viral vectors were associated with fundamental problems, including toxicity, immunogenicity, and limitations etc. Other most researches were to focus on designing non-viral vectors, such as cationic polymers that can form complexes with DNA to apply for gene delivery. Cationic lipids and polymers, the most important non-viral vectors, have many advantages over viral ones as non-immunogenic, easy to produce and not oncogenic.

However, the efficiency and toxicity are still obstacles to the application of non-viral vectors to gene therapy, for instance, transfection efficiency of PEI has been studied over a wide range of molecular weights, but high molecular weight polymers also result in significantly higher cytotoxicity.

The prior art taught the carriers, PEGylated colymers of _{L}-lysine and _{L}-phenylalanine, to deliver genes. The introduction of the hydrophilic PEG is believed to reduce the toxicity of the copolymer, due to its enhanced biocompatibility, and to impart improved stability to the complex under physiological conditions (Choi Y R et al. Development of polymeric gene delivery carriers: PEGylated copolymers of L-lysine and L-phenylalanine. J. Drug Targeting, 2007, 15, 391-398).

Also, Poly(D,L-Lactic-co-Glycolic Acid)(PLGA) is a well known non-virus carrier due to its hydrophilic of cell membrane affinity, high biocompatibility and biodegradability. The previous studies disclosed the cationic polymers modified by PLGA revealed better transfect efficiency and lower cytotoxicity (Gwak, S. J. et al. Poly(lactic-co-glycolic acid) nanosphere as a vehicle for gene delivery to human cord blood-derived mesenchymal stem cells: comparison with polyethylenimine. Biotechnol. Lett., 2008, 30, 1177-1182). In spite of the attempts and advances, there still continues to be a need to improve polymeric delivery platforms.

### SUMMARY OF THE INVENTION

The present invention provides a nanoparticle of polymer skeleton having a formula:

Y-A-PEG-X-PEG-A-Y

wherein X represents quaternization of amine moity;
A represents at least an amino acid; and
Y represents hydrophobic biopolymer.

The present invention also provides a therapeutic component delivery system comprising nanoparticles mentioned above and at least a therapeutic component.

### BRIEF DESCRIPTION OF THE DRAWINGS

FIG. 1 shows the synthesis scheme of Bis-(PLGA-Phe-PEG)-qDETA.

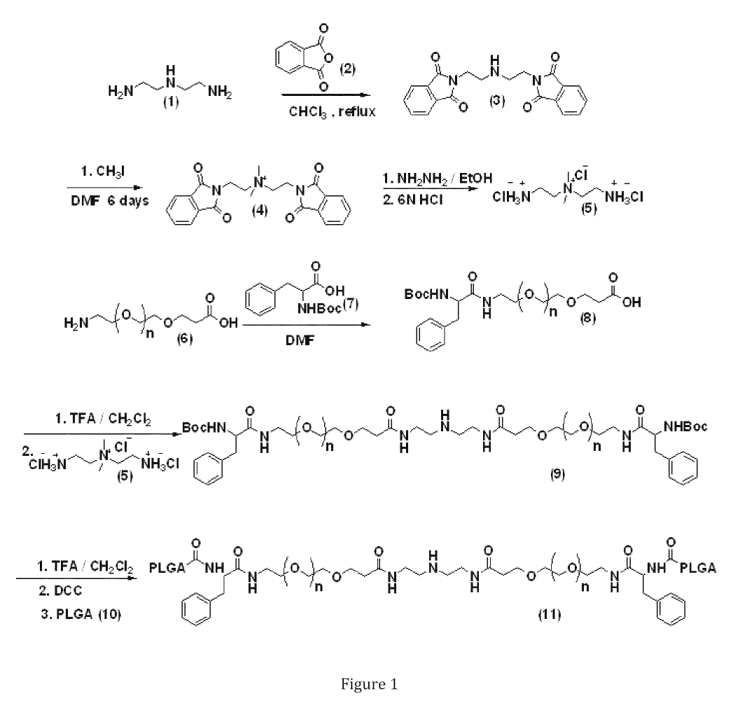

FIG. 2 shows cell bioviability rate in MTS assay.

FIG. 3 shows DNA gel retardation assay of cationic nanoparticles.

FIG. 4 shows GFP expression in 293T cells. The picture in the left side was shot by phase contrast microscopy, and the right side was shot by fluorescence microscope.

FIG. 5 shows the mineralization of hADSCs, which were treated in different kinds of drug and/or drug carriers.

### DETAILED DESCRIPTION OF THE INVENTION

The main objective of the present invention is the development of new cationic nanopolymer carrier system for the delivery of plasmid DNA (pDNA) and poorly water soluble drug (hydrophobic drug).

The present invention provides a nanoparticle of polymer skeleton having a formula:

Y-A-PEG-X-PEG-A-Y

wherein X represents quaternization of amine moity;
A represents at least an amino acid; and
Y represents hydrophobic biopolymer.

Y of the nanoparticle is phospholipids, lecithin, polylactic acid (PLA), polyglycolic acid (PGA), polylactic-co-glycolic acid (PLGA), polyglutamic acid (PGA), polycaprolactone (PCL), polyanhydrides, polyamino acid, polydioxanone, polyhydroxybutyrate, polyphophazenes, polyesterurethane, polycarbosyphenoxypropane-cosebacic acid or polyorthoester or a mixture thereof. In the embodiment of the invention, Y is polylactic-co-glycolic acid (PLGA).

The amino acid of the nanoparticle is hydrophobic amino acid which is selected from the group consisting of phenylalanine, isolucine, tryptophan, leucine, valine, and methionine. In particular, the amino acid is phenylalanine.

The nanoparticle disclosed herein is a cationic nanoparticle. The cationic nature of the cationic nanoparticles provides the nanoparticles with various advantages. For example, the cationic nature of nanoparticles allows the nanoparticles to ionically attach to negatively charged species, such as oligonucleotides, or to alter biodistribution.

In one embodiment, the nanoparticle has a size in a range from about 0.01 nm to about 1000 nm. In another embodiment, the nanoparticle has a size in a range from about 0.1 nm to about 600 nm. In yet another embodiment, the nanoparticle has a size in a range from about 1 nm to about 250 nm.

In one embodiment, the cationic nanoparticle has a zeta potential of 0.1-60 mV. In further embodiment, the cationic nanoparticle has a zeta potential of 1-30 mV. New amphiphilic muti-block cationic copolymer based on diethylenetriamine (DETA) monomer and quaternization of amine of DETA (qDETA) bring the positive charge to decrease toxicity. Therefore; the nanoparticle of the present invention is Bis-(PLGA-Phe-PEG)-qDETA which has structure:

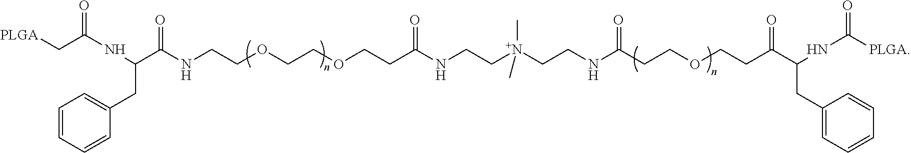

The Bis-(PLGA-Phe-PEG)-qDETA of new cationic copolymer was synthesized by quaternary ammonium moiety, PEG, phenylalanine and PLGA. Among them, PLGA has hydrophobicity, biodegradability and biocompatibility, PEG can be reduced immunity and prolonged the circulation time in vivo, and the phenylalanine which can be improved the hydrophobicity. Finally, new cationic nanoparticles have been prepared by water miscibility of solvent.

The present invention also provides a therapeutic component delivery system comprising a nanoparticle mentioned above and at least a therapeutic component. The nanoparticle and therapeutic component form a nanocomplex. The weight ratio of nanoparticles to therapeutic component is in the range of about 1:0.0001 to about 1:1. The N/P weight ratio of pDNA/Cationic-nanoparticles is about 6.25/1 showed an optimal zeta-potential and binding affinity than others, suggesting the lower toxicity of the pDNA/Cationic-nanoparticles.

The therapeutic component is hydrophobic drug, oligonucleotide, peptide, protein, antibody, or vaccine. In the embodiment of the invention, the therapeutic component is hydrophobic drug or oligonucleotide.

The oligonucleotide may be single or double-stranded, linear or circular, natural or synthetic, and without any size limitation. The oligonucleotide may be in the form of a plasmid or of viral DNA or RNA. Furthermore, the oligonucleotide may include modifications, such as phosphothioates or peptide nucleic acids (PNA). The RNA used herein comprises short inhibitory RNA, short hairpin RNA, micro RNA, and combinations thereof, and the DNA comprises double strand DNA, single strand DNA and combinations thereof.

Cytotoxicity and gene transfection efficiency of pDNA/Cationic-nanoparticles were tested in vitro using cell culture. The cationic-nanoparticles of Bis-(PLGA-Phe-PEG)-qDETA copolymers of the present invention showed higher gene transfection efficiency. These results indicate that new cationic copolymer is promising candidates for gene delivery vehicles or hydrophobic drug carrier.

### EXAMPLES

The examples below are non-limiting and are merely representative of various aspects and features of the present invention.

### Example 1

### Synthesis of Bis-(PLGA-PEG-Phe)-qDETA

### 1,5-Bis(phthalimido)-3-azapentane (3)

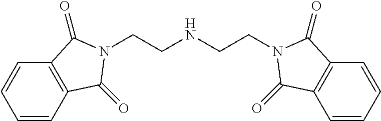

Phthalic anhydride (7 g, 47.3 mmole) was mixed well with chloroform (30 ml). Diethylenetriamine (2 g) was added slowly into the mixture, and refluxed the mixture for 5 hr. The product was dissolved in dichloromethane (25 ml) while it was cooling to room temperature. The solution was filtered and vacuum concentrated. After washing twice by ethanol (10 ml), the mixture was filtrated by column chromatography which was filled by silica gel and then eluted by ethyl acetate. An opal powder was obtained in 68% yield.

¹H NMR (CDCl3, 200 MHz) δ7.69-7.80 (m, 4H), δ 3.771 (t, 211), δ 2.950 (t, 2H). ESI-MS: calcd for C20H17N3O4: 363.12. Found 363.94.

### 2-(1,3-dioxoisoindolin-2-yl)-N-(2-(1,3-dioxoisoindolin-2-yl)ethyl)-N,Ndimethylethanaminium salt (4)

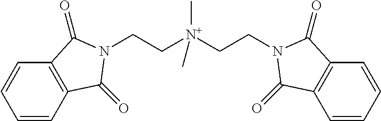

Compound (3) (100 mg, 0.275 mmole) was placed in a 100 ml rounded bottom flask with 5 ml DMF, then added into 1 ml CH₃I over 6 days. The product was extracted by methonal and centrifuged at 8000 rpm for 10 min in 38% yield.

¹H NMR (d6-DMSO, 200 MHz) δ7.879-7.908 (m, 4H), δ 4.067 (t, 2H), δ 3.717 (t, 2H), δ 3.240 (s, 3H). ESI-MS: calcd for C₂₂H₂₂N₃₀₄+:392.16. Found 391.95

### 2-amino-N-(2-aminoethyl)-N,N-dimethylethanaminium chloride salt (5)

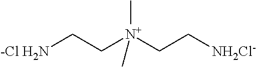

The mixture of compound (4) (100 mg, 0.254 mmole), ethanol (15 ml) and NH₂NH₂ (35%, 1.45 ml) was refluxed for 2 hr. The solution was filtered and added some HCl (2N). The solution was filtered again and added ethanol to extract the final product. After 10 min centrifuging (6000 rpm) and vacuum dring, the product was obtained in 80.38% yield.

¹H NMR (D2O, 200 MHz) δ3.697 (t, 2H), δ3.551 (t, 2H), δ3.227 (s, 3H). ESI-MS: calcd for C6H18N3+:132.23. Found 132.25

### tert-butyl-phenylanaline-PEG-COOH (8)

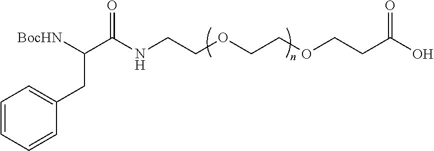

Boc-Phe-OH (7) (200 mg, 0.754 mmole) and DCC (450 mg, 2.18 mmole) were mixed and vacuum dried in a beaker. DMF (15 ml) was put into the beaker. After 4 hr, PEG (6) (300 ml, 0.088 mmole) was mixed with the previous prepared solution, and then TEA (0.1 ml) was added into the beaker. 2 hr later, the solution was filtered and dialyzed (MWC0=1000) by MeOH for 12 hr. After concentrating, the product (8) was extracted by ether.

¹H NMR (CDCl3, 200 MHz) δ3.642 (s, 33H, δ1.397 (s, 1H).

### Bis-(Boc-Phe-PEG)-qDETA (9)

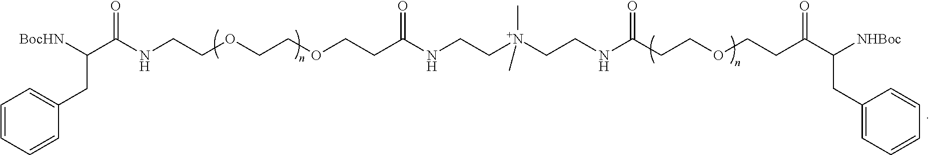

Compound (5) (70 mg, 0.53 mmole) was mixed with TEA (0.5 ml) and DMSO (5 ml) in a beaker. Compound (8) (100 mg, 0.0273 mmole) and DCC (70 mg, 0.340 mmole) were added in a new beaker and added DMSO (10 ml) after the mixture dried. 4 hr later, the solutions in two beakers were mixed. After overnight reaction, the solution was filtered and dialysis (MWCO=1000) by MeOH for 12 hr. After concentrating, the product (9) was extracted by ether. The white powder was obtained in 86% yield.

¹H NMR (CDCl3, 200 MHz) δ3.642 (s, 33H), δ3.453 (s, 1H), δ1.397 (s, 1H). GPC-MS: calcd for compound (9). Found Ww: 7051 (PDI: 1.023)

### Bis-(PLGA-Phe-PEG)-qDETA (11)

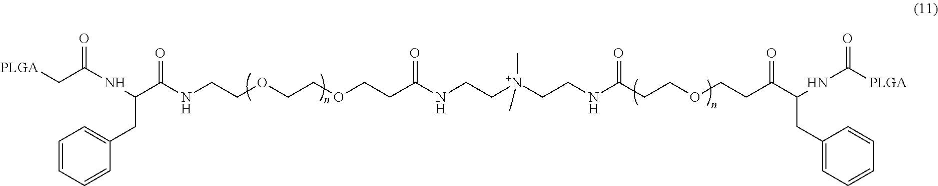

Compound (9) (80 mg, 0.0114 mmole) was dissolved in CF₃COOH/CH₂Cl2 (1:1, 3 ml) for 30 min and vacuum dried. The dried power was dissolved in CH₂Cl₂ (10 ml) and vacuum dried, and repeated this step three times. DMF (5 ml) was added to dissolve the dried powder. PLGA (1 g, 0.0016 mmole) and DCC (20 mg, 0.097 mmole) were dissolved in DMF (15 ml) in a new beaker. 4 hr later, the solutions in two beakers were mixed. After 2 hr reaction, the solution was filtered and dialysis (MWCO=1200) by DMF for 12 hr continuously. After concentrating and ether extracting, the powder was dissolved in CH₂Cl₂ and washed by MeOH (3 ml) twice. After drying, the final product (11) was obtained in 92% yield.

¹H NMR (CDCl3, 200 MHz) δ5.219 (m, 11H), δ4.667-4.817 (m, 22H),

δ3.459 (s, 1H), δ1.562 (m, 33H). GPC-MS: calcd for compound (11). Found Ww: 22742 (PDI: 1.656)

### Example 2

### Preparing and Analyzing Nanoparticles

The compound (11) (10 mg) was dissolved into DMF (1 ml), and then the cationic nanoparticles were prepared by water miscibility of solution. The average size of the nanoparticle detected by zetasizer is 177.1±8 nm.

Properties of the Cationic-nanoparticles were evaluated with particle size, zeta-potential and PEG antibiotic assay.

First, the zeta-potential and particle size of nanoparticles were examined in different concentration of compound (11). The serial concentration of compound (11) (5 mg, 10 mg, 25 mg and 50 mg) emulsified with DMF (1 ml), and the emulsified solution was detected by zetasizer respectively. The results in table 1 showed the size of nanoparticles were in range of 150˜250 nm and zeta potential were about −15˜20 mv. However, the zeta potential was negative while the concentration of compound (11) was under 10 mg. In order to produce cationic nanoparticles, the concentration of compound (11) had to be controlled.

TABLE 1
the particle zeta potential in the different
concentration of compound (11)

| Compound (11) | DMF | ddH₂O | Zeta potential | Ave. size |
|---|---|---|---|---|
| (mg) | (ml) | (ml) | (ζ, mV) | (nm) |
| 5 | 1 | 2 | −15 | 149.1 |
| 10 | 1 | 2 | 9.8 | 177.2 |
| 25 | 1 | 2 | 21 | 217.6 |
| 50 | 1 | 2 | 21.3 | 246.6 |

The PEG antibody, AGP3, was used for identified the structure of cationic nanoparticles prepared previously by ELISA. Three samples, NPs (cationic nanoparticles, 1 μg/ml), nanoparticles of compound (10) (negative control, 1 μg/ml) and PEG-lipopolex (positive control, commercial particles, 1 μg/ml), were detected. The absorbances of these samples were, measured at 410 nm, and the experiment was performed in triplicate. The result in table 2 revealed that the nanoparticles had PEG properties.

TABLE 2
The absorbance of three samples in triplicate

| 1 μg/ml | 1 | 2 | 3 |
|---|---|---|---|
| Nanoparticles of | 0.074 | 0.075 | 0.076 |
| compound (10) | | | |
| NPs (11) | 1.111 | 1.118 | 1.089 |
| PEG-lipopolex | 1.921 | 2.187 | 1.896 |

### Example 3

### Cytotoxicity of Nanoparticles

Cell viability was examined with MTS assay. For detection of the cytotoxicity of the nanoparticles, cells were cultured in 96-well plates and treated with the nanoparticles. Compound 11 (2 ng/100 μl) and PEI (commercial cationic particle, 10 kDa, 2 ng/100 μl) were dissolved in DMSO and diluted by medium separately. The diluted solution (100 μl/well) was added into the plate. After 24 hours incubation, the medium was replaced with 200 ul fresh medium, and 20 ul of CellTiter 96 Aqueous was added to the each well. Then, incubate the microplate at 37° C. for two hours and record absorbance at 490 nm with a 96-well plate reader.

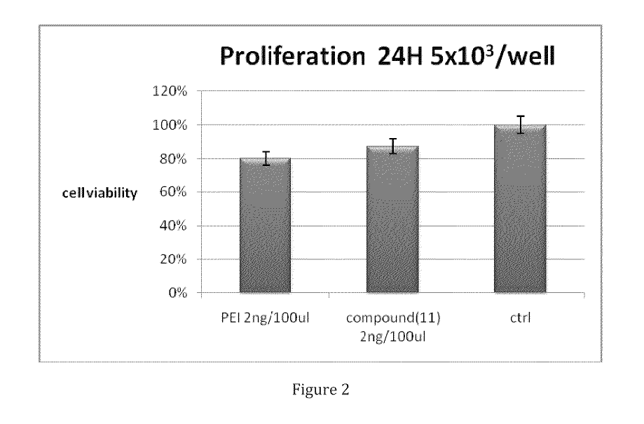

As FIG. 2 showed, the mitochondrial activity of the cells of compound II treated cell is higher (87%) than PEI treated cell (80%) after 24 hr incubation. Therefore, it was proved that the copolymer in the present invention has no significant cytotoxicity.

### Example 4

### Gel Retardation Assay

Different amounts of cationic nanoparticle were mixed well with plasmid DNA (0.5 ug, pEGFP-N1) in room temperature for 10 min. The effect of cationic nanoparticle on condensation of DNA was investigated using electrophoresis on 0.8% agarose gel with TAE running buffer at 100 V for 40 min.

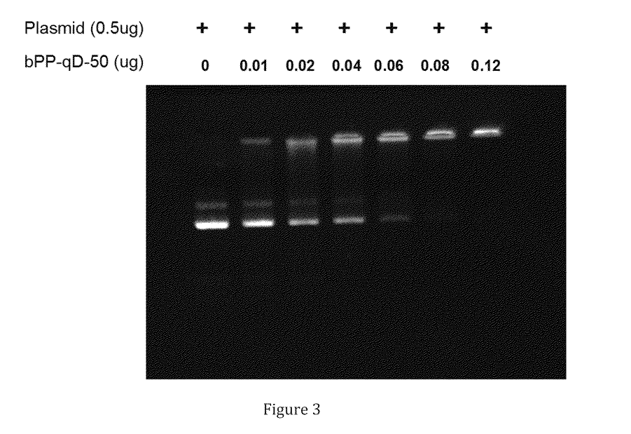

The result of the gel retardation assay showed that the N/P weight ratio of cationic nanoparticles/DNA was greater than about 6.25 (0.08 μg nanoparticle and 0.5 μg DNA), cationic nanoparticles/DNA complex bound tightly and completely (FIG. 3).

### Example 5

### Transfection Efficiency of the Cationic Nanoparticles

For evaluating transfection efficiency, 293T cells (human embryonic kidney cell) were culture in DEME medium with 10% BSA. The cationic nanoparticle/DNA complex was prepared by mixing 0.5 μg of pEGFP-N1 with the same amounts of cationic nanoparticles for 10 min. Different amounts of complex were mixed with serum-free DEME medium, and the final volume was adjusted to 250 μl. The complex solution was mixed with 293T cells and incubated for 4˜6 hr. Then 1 ml of serum-free medium was replaced with fresh medium containing serum After 48 hr, arrays were observed with a fluorescence inverted microscope.

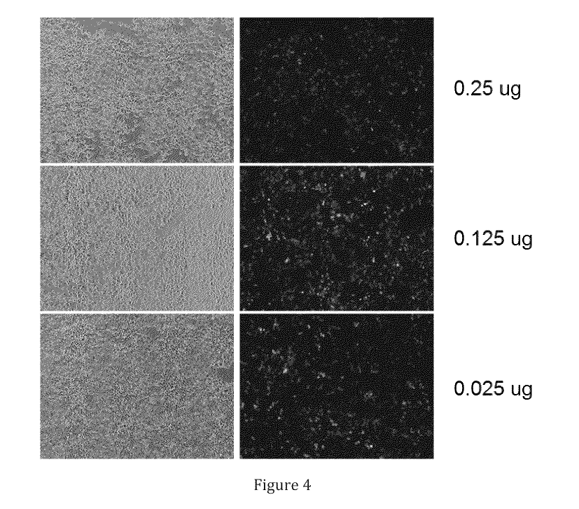

The transfection effect of the nanoparticle in the present invention was observed using fluorescence microscopy. As FIG. 4 shown, the complex containing 0.125 μg of nanoparticle had better transfection efficiency. It proved that the nanoparticle was the potential candidate of gene delivery.

### Example 6

### Nanoparticle for Hydrophobic Drug Delivery

TCH-2972, a new hydrophobic drug of Isoflavonoid derivatives for inhibiting bone resorption and preventing osteoporosis, was used for identifying the ability of delivering hydrophobic drug of the nanoparticle (U.S. Pat. No. 7,618,998 B2). hADSCs (Human Adipose-Derived Stem Cells) was cultured in 48-well plate for 24 hr, and then TCH-2972, carrier (nanoparticles) and nanoparticle/TCH-2972 complex were treated the hADSCs respectively. After 72 hr, the medium was replaced. 10 days later, Alizarin Red S, an anthraquinone derivative, was used to identify mineralization of hADSCs.

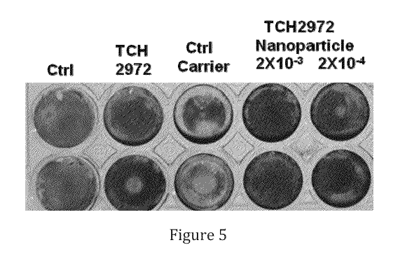

As FIG. 5 shown, nanoparticle/TCH-2972 complex treated cells had great ossification ability than the cells treated TCH-2972 solely. Also, the result demonstrated that the ossification ability increased due to the rising of transfer rate rather than the nanoparticle itself.

One skilled in the art readily appreciates that the present invention is well adapted to carry out the objects and obtain the ends and advantages mentioned, as well as those inherent therein. The nanoparticles, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A nanoparticle of polymer skeleton having a formula:
Y-A-PEG-X-PEG-A-Y
wherein X represents quaternization of amine moiety;
A represents at least an amino acid; and
Y represents hydrophobic biopolymer.

2. The nanoparticle of claim 1, wherein Y is phospholipids, lecithin, polylactic acid (PLA), polyglycolic acid (PGA), polylactic-co-glycolic acid (PLGA), polyglutamic acid (PGA), polycaprolactone (PCL), polyanhydrides, polyamino acid, polydioxanone, polyhydroxybutyrate, polyphophazenes, polyesterurethane, polycarbosyphenoxypropane-cosebacic acid or polyorthoester or a mixture thereof.

3. The nanoparticle of claim 2, wherein Y is polylactic-co-glycolic acid (PLGA).

4. The nanoparticle of claim 1, wherein the amino acid is hydrophobic amino acid.

5. The nanoparticle of claim 4, wherein the amino acid is selected from the group consisting of phenylalanine, isolucine, tryptophan, leucine, valine, and methionine.

6. The nanoparticle of claim 5, wherein the amino acid is phenylalanine.

7. The nanoparticle of claim 1, which is a cationic nanoparticle.

8. The nanoparticle of claim 1, wherein the nanoparticle has a size in a range from about 0.1 nm to about 600 nm.

9. The nanoparticle of claim 8, wherein the nanoparticle has a size in a range from about 1 nm to about 250 nm.

10. The nanoparticle of claim 1, wherein the nanoparticle has a zeta potential in a range from about 0.1 mV to about 60 mV.

11. The nanoparticle of claim 10, wherein the nanoparticle has a zeta potential in a range from about 1 mV to about 30 mV.

12. The nanoparticle of claim 1, which is

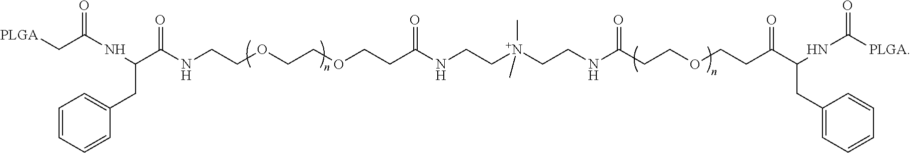

13. A therapeutic component delivery system comprising a nanoparticle of claim 1 and at least a therapeutic component, wherein the nanoparticle is a cationic particle.

14. The system of claim 13, wherein a ratio of the weight of nanoparticle compared to the weight of therapeutic compound lies in the range from about 1:0.0001 to about 1:1.

15. The system of claim 13, wherein the therapeutic component is hydrophobic drug, oligonucleotide, peptide, protein, antibody, or vaccine.

16. The system of claim 15, wherein the therapeutic component is hydrophobic drug or oligonucleotide.

17. The system of claim 15, wherein the oligonucleotide is RNA and DNA.

18. The system of claim 17, wherein the RNA comprises short inhibitory RNA, short hairpin RNA, micro RNA, and combinations thereof.

19. The system of claim 17, wherein the DNA comprises double strand DNA, single strand DNA, plasmid DNA, and combinations thereof.
